(19) United States
(12) Patent Application Publication
(10) Pub. No.: US 2022/0326156 A1
(43) Pub. Date: Oct. 13, 2022

(54) ADAPTABLE ILLUMINATION PATTERN FOR SAMPLE ANALYSIS

(71) Applicant: Illumina, Inc., San Diego, CA (US)

(72) Inventors: Simon Prince, Carlsbad, CA (US); Krysada Phounsiri, Chula Vista, CA (US); Wenyi FENG, San Diego, CA (US)

(21) Appl. No.: 17/658,238

(22) Filed: Apr. 6, 2022

Related U.S. Application Data
(60) Provisional application No. 63/200,982, filed on Apr. 7, 2021.

(51) Int. Cl. G01N 21/64

(52) U.S. Cl. CPC G01N 21/6428; G01N 21/6458; G01N 21/6454

(57) ABSTRACT

A system for analysis of a sample at a substrate comprises: a light source to generate first light; and a spatial light modulator to form second light from the first light, wherein the substrate includes at least one sensor to detect an emission emitted based on the second light, wherein at the substrate the second light forms a shape selected based on the at least one sensor, wherein the second light illuminates an area of the substrate corresponding to the shape.

### CROSS-REFERENCE TO RELATED APPLICATION

This application claims priority to U.S. Patent Application No. 63/200,982, filed on Apr. 7, 2021, and entitled “ADAPTABLE ILLUMINATION PATTERN FOR SAMPLE ANALYSIS,” the disclosure of which is incorporated by reference herein in its entirety.

### BACKGROUND

Samples of different materials can be analyzed using one or more of a variety of analytical processes. For example, sequencing such as high-throughput DNA sequencing can be the basis for genomic analysis and other genetic research. In this and other types of analysis, characteristics of a sample can be determined by illuminating the sample, and by detecting emissions (e.g., fluorescent light) that is generated in response to the illumination.

### SUMMARY

In a first aspect, a system for analysis of a sample at a substrate comprises: a light source to generate first light; and a spatial light modulator to form second light from the first light, wherein the substrate includes at least one sensor to detect an emission emitted based on the second light, wherein at the substrate the second light forms a shape selected based on the at least one sensor, wherein the second light illuminates an area of the substrate corresponding to the shape.

Implementations can include any or all of the following features. The spatial light modulator is a transmissive spatial light modulator. The spatial light modulator is a reflective spatial light modulator. The spatial light modulator is an amplitude spatial light modulator. The amplitude spatial light modulator includes mirrors that are orientable, and wherein the amplitude spatial light modulator orients at least a first mirror of the mirrors to form the second light. The system further comprises a beam dump, wherein the amplitude spatial light modulator orients at least a second mirror of the mirrors to direct third light at the beam dump, the third light being part of the first light. The spatial light modulator is a phase spatial light modulator. The area of the substrate includes a polygon. The second light illuminates multiple areas of the substrate, each of the multiple areas being a polygon area. The area of the substrate includes an ellipse. The second light illuminates multiple areas of the substrate, each of the multiple areas being an ellipse area. The second light illuminates multiple areas of the substrate. At least two of the multiple areas have different shapes from each other. The sensor comprises a complementary metal oxide semiconductor device. The substrate includes a flow cell for the sample. The substrate includes multiple sensors covered by the flow cell. The substrate includes multiple flow cells. The substrate includes multiple sensors, and wherein each of the multiple flow cells covers at least a corresponding one of the multiple sensors. The substrate further includes an identifier relating to the sensor, wherein the system detects the identifier at the substrate, and wherein the shape is selected based on the detected identifier. The identifier comprises at least one of a radiofrequency identification tag or a visual code. The emission comprises fluorescent light.

In a second aspect, a method comprises: directing first light at a spatial light modulator in a system configured for analysis of a sample at a substrate, wherein the substrate includes at least one sensor; controlling the spatial light modulator to form second light from the first light; directing the second light at the substrate, wherein the second light illuminates an area of the substrate having a shape selected based on the at least one sensor; and detecting an emission emitted based on the second light.

Implementations can include any or all of the following features. The spatial light modulator is an amplitude spatial light modulator, wherein the amplitude spatial light modulator includes mirrors that are orientable, and wherein controlling the amplitude spatial light modulator to form the second light comprises orienting at least a first mirror of the mirrors. The first mirror is oriented to assume one of at least two states. The method further comprises orienting at least a second mirror of the mirrors to direct third light at a beam dump, the third light being part of the first light. The spatial light modulator is a phase spatial light modulator, and wherein controlling the phase spatial light modulator to form the second light comprises changing an intensity profile or an irradiance profile of the second light. Changing the intensity profile or the irradiance profile comprises changing the second light from illuminating a plurality of areas of the substrate to illuminating at least one area that is fewer than the plurality of areas, and wherein a brightness is increased in the at least one area. The second light illuminates multiple areas of the substrate. At least two of the multiple areas have different shapes from each other. The substrate further includes an identifier relating to the sensor, the method further comprising detecting, by the system, the identifier at the substrate, and wherein the shape is selected based on the detected identifier. Detecting the identifier comprises at least one of receiving a signal from a radiofrequency identification tag, or reading a visual code. The method further comprises providing real-time feedback regarding the second light; and controlling the spatial light modulator using the real-time feedback. Providing the real-time feedback comprises: creating an inverse of an imaging pattern of the second light; and multiplying the inverse with imaging obtained at the substrate. The emission comprises fluorescent light.

### BRIEF DESCRIPTION OF DRAWINGS

FIG. 1 is a diagram of a system including an instrument, a cartridge, and a flow cell.

FIG. 2 is a schematic view of an example system that can be used for biological and/or chemical analysis.

FIG. 3 shows an example of a system.

FIG. 4 shows an example of a system with a spatial light modulator.

FIG. 5 shows an example of a system with an amplitude spatial light modulator.

FIG. 6 shows an example of a system with a phase spatial light modulator.

FIG. 7 shows an example of a system with real-time feedback to a spatial light modulator.

FIG. 8 shows an example of a substrate having a flow cell and sensors.

FIG. 9 shows an example of a substrate having flow cells and sensors.

FIG. 10 shows an example of a method.

FIG. 11 illustrates an example architecture of a computing device 1100 that can be used to implement aspects of the present disclosure, including any of the systems, apparatuses, and/or techniques described herein, or any other systems, apparatuses, and/or techniques that may be utilized in the various possible embodiments.

### DETAILED DESCRIPTION

This document describes examples of systems and techniques for providing an adaptive illumination pattern for performing analysis of a sample at a substrate. For example, the present subject matter can generate one or more illumination footprints at the substrate for sample analysis. The illumination pattern can be adapted to accommodate the particular number of sensors (e.g., one or more) that the substrate includes for imaging of the sample, and/or for the size or position of such sensor(s). In some implementations, the illumination pattern can instead or additionally be adapted for emission detection by one or more sensors not included in the substrate.

Examples described herein refer to imaging. Imaging can be performed to analyze a sample of any of multiple materials. In some implementations, one or more types of imaging can be performed as part of biological analysis of a biological material, or chemical analysis of any material. For example, a process of sequencing genetic material can be performed. In one example, the process can be a DNA sequencing process, e.g., sequencing-by-synthesis or next-generation sequencing (also known as high-throughput sequencing). In another example, the process may be used to enable genotyping. Genotyping involves determining differences in the genetic make-up (genotype) of an individual by examining the individual's DNA sequence using biological assays and comparing it to another individual's sequence or a reference sequence. Such processes can involve fluorescent imaging, where a sample of genetic material is subjected to excitation light (e.g., a laser beam) to trigger a fluorescent emission response by one or more markers associated with the genetic material. Some nucleotides can have fluorescent tags applied to them and which pair with a complementary nucleotide of the sample genetic material. The fluorescent tags can fluoresce responsive to exposure to an excitation energy source. One or more wavelength spectra of the fluorescent emission response can be used to determine the presence of a corresponding nucleotide. Fluorescent emission responses can be detected over the course of the sequencing process and used to build a record of nucleotides in the sample.

Examples described herein refer to a spatial light modulator. As used herein, a spatial light modulator modifies at least one of an amplitude of light, or a phase of light, in order to control a spatial characteristic of the light. A spatial light modulator can be reflective (e.g., performing light modification by way of reflection) or transmissive (e.g., performing light modification by way of transmission). Any of multiple technologies for spatial light modulators can be used. In some implementations, a spatial light modulator uses orientable mirrors. In some implementations, a spatial light modulator uses liquid crystals. In some implementations, a spatial light modulator uses an active-matrix technology (e.g., liquid crystal on silicon). In some implementations, collimated light can be provided to any spatial light modulator mentioned herein. For example, illumination light can be passed through at least one collimator before reaching the spatial light modulator.

Examples described herein refer to substrates. A substrate may refer to any material that provides a substantially rigid structure, or to a structure that retains its shape rather than taking on the shape of a vessel to which it is placed in contact. The material can have a surface to which another material can be attached including, for example, smooth supports (e.g., metal, glass, plastic, silicon, and ceramic surfaces), as well as textured and/or porous materials. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, resins, silica or silica-based materials including silicon and modified silicon, carbon, metals, inorganic glasses, plastics, optical fiber bundles, and a variety of other polymers. In general, the substrates allow optical detection and do not themselves appreciably fluoresce.

Examples herein refer to a substrate that includes a sensor. As used herein, a substrate can include one or more sensors designed to detect an event, property, quality, or characteristic. On-chip imaging may involve configurations where an imaging substrate is to be situated on-chip relative to the image sensor(s). For example, this can reduce, and in some instances even eliminate, the use of emission optics between the sample and the image sensor, such emission optics including, but not limited to, objective(s), lens(s), and filter(s). Any of multiple types of sensor technology can be used. In some implementations, metal-oxide-semiconductor (MOS) devices can be used with on-chip imaging. For example, a complementary MOS (CMOS) device (e.g., a CMOS chip) can be used. In some implementations, a charge-coupled device (CCD) is used with on-chip imaging. Other types of sensors can be used, additionally or alternatively.

Examples described herein refer to flow cells. A flow cell is a substrate that can be used in preparing and accommodating or carrying one or more samples in at least one stage of an analysis process. The flow cell is made of a material that can be usable with both the sample genetic material and the illumination and chemical reactions to which it will be exposed. The substrate can have one or more channels in which sample genetic material can be deposited. A substance (e.g., a liquid) can be flowed through the channel where the sample genetic material is present to trigger one or more chemical reactions and/or to remove unwanted material. The flow cell may enable the imaging by facilitating that the sample in the flow cell channel can be subjected to illuminating excitation light and that any fluorescent emission responses from the sample can be detected. Some implementations may be designed to be used with at least one flow cell, but may not include the flow cell(s) during one or more stages, such as during shipping or when delivered to a customer. For example, the flow cell(s) can be installed into an implementation at the customer's premises in order to perform analysis.

The examples described herein can provide advantages compared to previous approaches. Flexibility, extensibility, and/or throughput of analysis operations can be improved. Illumination in an analysis system can be dynamically and automatically reconfigured based on the type of sensor(s) in the substrate holding the sample. Brightness of excitation light (e.g., an intensity profile or an irradiance profile) can be flexibly controlled by the analysis system. Lowering of a uniformity requirement for illumination light in analysis system can be facilitated. The spatial dimensions of illumination light can be controlled to reduce or eliminate inadvertent illumination of other circuitry (e.g., non-sensor circuitry) at the substrate. An analysis system can be designed so as to flexibly adapt to later technology advances in sensor technology.

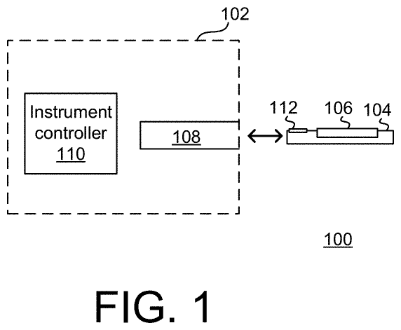

FIG. 1 is a diagram of a system 100 including an instrument 102, a cartridge 104, and a sample 106. The system 100 can be used for biological and/or chemical analysis, to name just two examples. The system 100 can be used together with, or in the implementation of, one or more other examples described elsewhere herein. Systems and/or techniques described herein can be part of the system 100 in some implementations.

The cartridge 104 can serve as a carrier for one or more instances of the sample 106. The cartridge 104 can be configured to hold the sample 106 and transport the sample 106 into and out of direct interaction with the instrument 102. For example, the cartridge 104 can be referred to as, or can include, a flow cell. The instrument 102 includes a receptacle 108 (e.g., an opening in its outer enclosure) to receive and accommodate the cartridge 104 at least during gathering of information from the sample. The cartridge 104 can be made of any suitable material(s). In some implementations, the cartridge 104 includes molded plastic or other durable material. For example, the cartridge 104 can form a frame for supporting or holding the sample 106.

The cartridge 104 can include one or more substrates configured to provide the sample 106 for analysis by the instrument 102. Any suitable material can be used for the substrate, including, but not limited to, glass, acrylic, and/or another plastic material. The cartridge 104 can facilitate that liquids or other fluids are selectively flowed relative to the sample 106. In some implementations, the cartridge 104 includes one or more flow structures for the sample 106. In some implementations, the cartridge 104 can include at least one flow channel. For example, a flow channel can include one or more fluidic ports to facilitate flow of fluid. For example, the sample 106 can be contained by a flow cell (e.g., as exemplified below).

The instrument 102 can operate to obtain any information or data that relates to at least one biological and/or chemical substance. The operation(s) can be controlled by a central unit or by one or more distributed controllers. Here, an instrument controller 110 is illustrated. For example, the instrument controller 110 can be implemented using at least one processor, at least one storage medium (e.g., a memory and/or a drive) holding instructions for the operations of the instrument 102, and one or more other components, for example as described in the following. In some implementations, the instrument 102 can perform optical operations, including, but not limited to, illumination and/or imaging of the sample(s). For example, the instrument 102 can include one or more optical subsystems (e.g., an illumination subsystem and/or an imaging subsystem). In some implementations, sensing of one or more emissions from (or associated with) the sample 106 (e.g., fluorescent light being generated by a fluorescent tag in response to excitation light) can be performed at least in part by the cartridge 104. For example, the cartridge 104 can include a sensor (e.g., as exemplified below) configured to detect one or more types of emissions from (or associated with) the sample 106. In some implementations, sensing of one or more emissions from (or associated with) the sample 106 (e.g., fluorescent light being generated by a fluorescent tag in response to excitation light) can be performed at least in part by the instrument 102. For example, the instrument 102 can include a sensor configured to detect one or more types of emissions from (or associated with) the sample 106. In some implementations, the instrument 102 can perform thermal treatment, including, but not limited to, thermal conditioning of the sample(s). For example, the instrument 102 can include one or more thermal subsystems (e.g., a heater and/or cooler). In some implementations, the instrument 102 can perform fluid management, including, but not limited to, adding and/or removing fluid in contact with the sample(s). For example, the instrument 102 can include one or more fluid subsystems (e.g., a pump and/or a reservoir).

The cartridge 104 can include an identifier 112 relating to the sensor(s) of the cartridge 104. In some implementations, the identifier 112 can indicate at least the number of sensors (e.g., one or more) that the cartridge 104 includes. The instrument 102 can detect the identifier 112 and select a shape of the illumination light (e.g., excitation light) based on the sensor(s). For example, when the identifier 112 indicates that the cartridge 104 includes one sensor, the instrument 102 can generate illumination light in one area; when there are two sensors in the cartridge 104, two areas of illumination light can be generated, and so on. In some implementations, the identifier 112 can be provided using any of multiple technologies. For example, the identifier 112 can include a radiofrequency identification (RFID) tag. As another example, the identifier 112 can include a visual code.

In some implementations, the identifier 112 may not relate to any sensor(s) of the cartridge 104. For example, the cartridge 104 may not include any sensor, and/or the instrument 102 may include one or more sensors to detect the emission(s). The identifier 112 can indicate a setting or other characteristic to be applied to such sensor(s) in the instrument 102. The identifier 112 can indicate how many areas of the sensor(s) should be used for detection, and/or the position of such area(s) at the sensor(s), to name just a few examples.

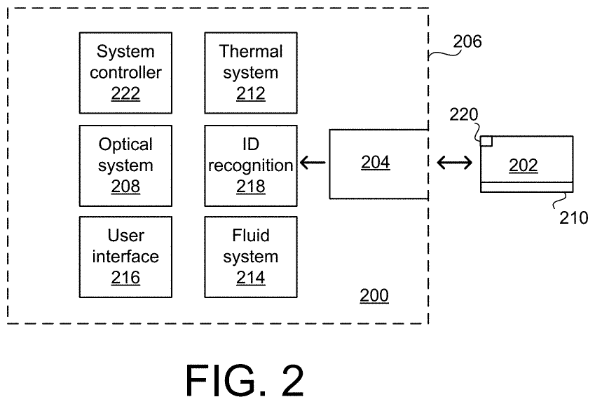

FIG. 2 is a schematic view of an example system 200, such as those described herein, that can be used for biological and/or chemical analysis, to name just two examples. Systems and/or techniques described herein can be part of the system 200 in some implementations. The system 200 can operate to obtain any information or data that relates to at least one biological and/or chemical substance. In some implementations, a carrier 202 supplies material to be analyzed. For example, the carrier 202 can include a substrate holding the material. In some implementations, the system 200 has a receptacle 204 to receive the carrier 202 at least during the analysis. The receptacle 204 can form an opening in a housing 206 of the system 200. For example, some or all components of the system 200 can be within the housing 206.

The system 200 can include an optical system 208 for biological and/or chemical analysis of the material(s) of the carrier 202. The optical system 208 can perform one or more optical operations, including, but not limited to, illumination and/or imaging of the material(s). For example, the optical system 208 can include any or all optical systems described elsewhere herein. As another example, the optical system 208 can perform any or all operations described elsewhere herein. In some implementations, the carrier 202 includes one or more sensors 210 to detect emissions from (or associated with) the material(s). For example, the optical system 208 may then be used for providing excitation light to the material(s). The present position of the sensor 210, being at the “bottom” of the carrier 202, is shown for illustrative purposes only. In some implementations, the optical system 208 can include one or more sensors to detect emissions from (or associated with) the material(s) at the carrier 202. When the optical system 208 includes multiple sensors, the sizing and/or positioning of the excitation illumination can be controlled based on a detection relating to the carrier 202. For example, the excitation illumination can be controlled based on the detected presence of a single or multiple sample areas (e.g., at the same or different substrates of the carrier 202).

The system 200 can include a thermal system 212 for providing thermal treatment relating to biological and/or chemical analysis. In some implementations, the thermal system 212 thermally conditions at least part of the material(s) to be analyzed and/or the carrier 202.

The system 200 can include a fluid system 214 for managing one or more fluids relating to biological and/or chemical analysis. In some implementations, the fluid(s) can be provided for the carrier 202 or its material(s). For example, fluid can be added to and/or removed from the sample material of the carrier 202.

The system 200 includes a user interface 216 that facilitates input and/or output relating to biological and/or chemical analysis. The user interface can be used to specify one or more parameters for the operation of the system 200 and/or to output results of biological and/or chemical analysis, to name just a few examples. For example, the user interface 216 can include one or more display screens (e.g., a touchscreen), a keyboard, and/or a pointing device (e.g., a mouse or a trackpad).

The system 200 can include an identifier recognition component 218 that facilitates detection of an identifier 220 at the carrier 202. In response to such detection, the optical system 208 can select a shape of the illumination light (e.g., excitation light) based on the sensor(s). In some implementations, the identifier 220 can include an RFID tag, and/or a visual code (e.g., in plain text or a symbolic representation), to name just a few examples. For example, the identifier recognition component 218 can include a wireless receiver for the RFID tag, and/or a scanner for the visual code.

In some implementations, the identifier 220 may not relate to any sensor(s) of the carrier 202. For example, the carrier 202 may not include any sensor, and/or the system 200 (e.g., the optical system 208) may include one or more sensors to detect the emission(s). The identifier 220 can indicate a setting or other characteristic to be applied to such sensor(s) in the system 200. The identifier 220 can indicate how many areas of the sensor(s) should be used for detection, and/or the position of such area(s) at the sensor(s), to name just a few examples.

The system 200 can include a system controller 222 that can control one or more aspects of the system 200 for performing biological and/or chemical analysis. The system controller 222 can control, and/or receive one or more signals from, one or more of the receptacle 204, the sensor 210, the optical system 208, the thermal system 212, the fluid system 214, the user interface 216, and/or the identifier recognition component 218. The system controller 222 can include at least one processor and at least one storage medium (e.g., a memory) with executable instructions for the processor.

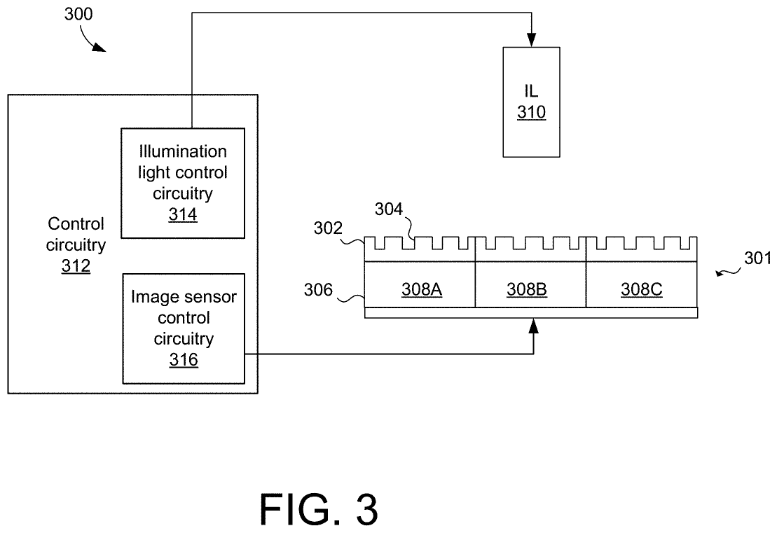

FIG. 3 shows an example of a system 300. The system 300 can be used with one or more other examples described elsewhere herein. The system 300 illustrates an example of sample analysis where the substrate holding the sample includes a sensor for detecting emissions. The system 300 includes an analysis substrate 301 that comprises a localization layer 302. The term localization is here used to illustrate that one or more aspects of a sample will be localized (e.g., have its exact or approximate location determined) relative to the localization layer 302. The localization layer 302 can include one or more features relating to sample position and/or confinement of electromagnetic radiation. For example, the localization layer 302 here includes cavities 304. In some implementations, the cavities 304 comprise nanowells. For example, nanowells can be formed by nanoimprinting lithography that uses a nanoscale template to form nanostructures in an imprinting resin. In some implementations, the cavities 304 comprise zero-mode waveguides (e.g., observation volumes of less than one nanoliter formed by optical confinements at a surface density exceeding 4*10⁴ confinements per mm²). The sample(s) at the localization layer 302 can be labeled with fluorescent dyes to be activated by excitation light. The present position of the localization layer 302, being at the “top” of the analysis substrate 301, is shown for illustrative purposes only.

The analysis substrate 301 comprises a sensor layer 306 that includes multiple sensor pixels. The sensor pixels of the sensor layer 306 can be part of one or more individual sensors, for example as described elsewhere herein. In this example, sensor pixels 308A-308C are shown for illustrative purposes. Any number of sensor pixels can be used. The sensor layer 306 can include a two-dimensional array of sensor pixels (e.g., a rectangular area with rows and columns of sensor pixels), of which the sensor pixels 308A-308C are shown in the present view. Each of the sensor pixels 308A-308C is sensitive to one or more forms of light (including, but not limited to, visible light). The sensor pixels 308A-308C can include, or be part of, one or more types of circuitry that facilitates detection of impinging electromagnetic radiation. In some implementations, one or more of the sensor pixels 308A-308C includes a photodiode. For example, the photodiode can include a junction between two types of semiconductor materials (e.g., a p-n junction). In some implementations, one or more of the sensor pixels 308A-308C is part of a chip of MOS devices. For example, one or more of the sensor pixels 308A-308C can be a CMOS device for detecting electromagnetic radiation. For example, the sensor layer 306 can comprise a CMOS chip. In some implementations, one or more of the sensor pixels 308A-308C includes a CCD. For example, one or more of the sensor pixels 308A-308C includes a MOS capacitor. In some implementations, the system 300 can use one or more sensors located elsewhere than at the substrate 301.

The sensor layer 306 can be configured for, or otherwise be compatible with, detecting emissions resulting from sample illumination with at least one type of excitation light. In some implementations, one or more colors of illumination light can be used. The illumination light can have one color, two colors, three colors, four colors, or more than four colors, to name just a few examples. For example, a red/green or blue/green color system can be used.

The sensor layer 306 can generate one or more corresponding output signals based on the detection by one or more of the sensor pixels 308A-308C. For example, the signal(s) can represent an image of the sample at the localization layer 302. In some implementations, the system 300 does not include a spacer between the localization layer 302 and the sensor layer 306.

The system 300 includes an illumination light (IL) source 310. In some implementations, the illumination light source 310 is directed toward the localization layer 302 for purposes of performing analysis. For example, the illumination light source 310 can include one or more lasers and/or one or more light-emitting diodes (LEDs).

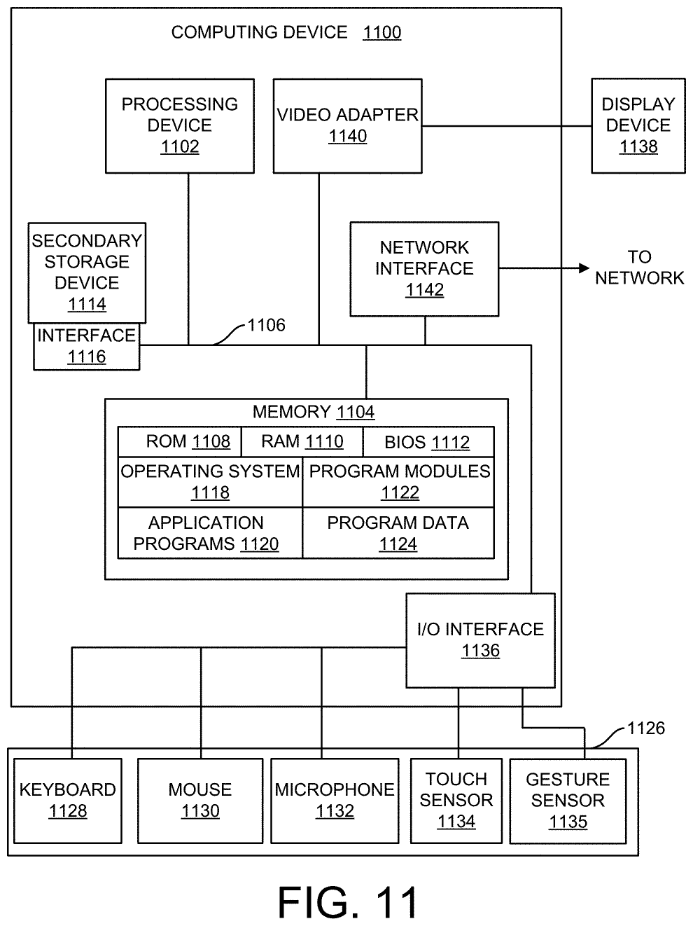

The system 300 includes control circuitry 312. The control circuitry 312 can be implemented using one or more examples described with reference to FIG. 11, and can be formed as a single unit or distributed among two or more components. The control circuitry 312 can include illumination light control circuitry 314 and image sensor control circuitry 316. The illumination light control circuitry 314 can generate illumination light using the illumination light source 310 (e.g., by controlling the wavelength range, intensity, duration, amplitude, and/or phase of the illumination light). The image sensor control circuitry 316 can provide for emission detection (e.g., by controlling an array of sensor pixels such as the sensor pixels 308A-308C in response to the sample being subjected to the illumination light).

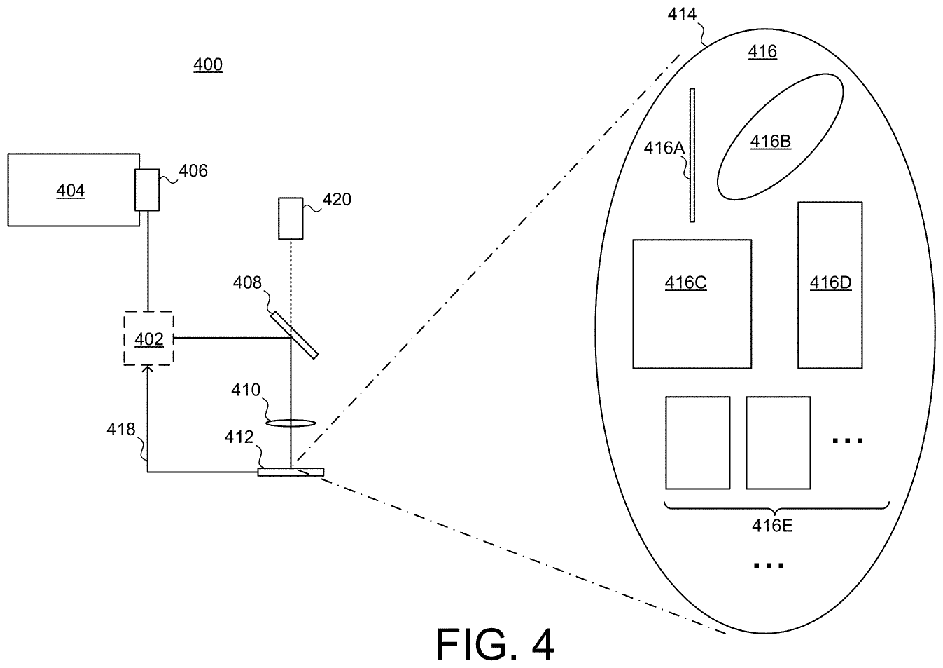

FIG. 4 shows an example of a system 400 with a spatial light modulator 402. The system 400 and/or the spatial light modulator 402 can be used with one or more other examples described elsewhere herein. The system 400 includes a light source 404. The light source 404 can generate one or more types of illumination light for analyzing a sample. For example, the light source 404 can be laser based or LED based. The system 400 can include optics 406 for the light source 404. For example, the optics 406 can include one or more lenses, one or more dichroic filters, and/or one or more collimators.

The optics 406 can provide light from the light source 404 to the spatial light modulator 402. The spatial light modulator 402 can receive first light generated by the light source 404 and form second light from the first light. In some implementations, the second light can include an amplitude modification compared to the first light. In some implementations, the second light can include a phase shift compared to the first light.

The spatial light modulator 402 can provide light (e.g., the second light in the above example) to one or more components in the system 400. Here, a mirror 408 and a lens 410 are shown as examples of components that can receive some or all of the light from the spatial light modulator 402. Other approaches can be used.

The system 400 can direct light from the spatial light modulator 402 to a substrate 412 that is configured for holding at least one sample for analysis. The substrate 412 includes at least one sensor for detecting the emission(s) from (or associated with) the sample. In some implementations, the substrate 412 includes one or more flow cells. In some implementations, the system 400 includes one or more sensors located elsewhere than at the substrate 412.

The light from the spatial light modulator 402 can form one or more shapes at the substrate 412. Here, an oval 414 schematically shows examples of shapes 416 that can be generated at the substrate 412. In these examples, each of the shapes 416 includes a polygon area or an ellipse area. A shape 416A includes a relatively narrow rectangle; a shape 416B includes an ellipse; a shape 416C includes a square; a shape 416D includes a rectangle wider than the one of the shape 416A; and shapes 416E includes two or more polygons (e.g., rectangles). The shapes 416 for the system 400 can include other shapes and/or sizes of shapes than the ones shown. One or more of the shapes 416 can be formed at the substrate 412. Two or more of the shapes 416 can be simultaneously and/or sequentially formed at the substrate 412. As such, the light from the spatial light modulator 402 can illuminate one or multiple areas of the substrate 412. At least two of such multiple areas can have different shapes from each other. Other shapes than those shown in this example can be used in some implementations.

In some implementations, the light impinging at the substrate 412 can be characterized by its cross-section area. For example, any of the shapes 416 can be described in terms of the area of the substrate that is illuminated based on such incoming light (e.g., whether the impinging light is a polygon or an ellipse; whether the impinging light is relatively narrow or wide; and/or whether the impinging light forms one or more illuminated areas). One or more spatial dimensions (e.g., width, length, height, depth, etc.) of the impinging light can be specified. With a phase spatial light modulator, the impinging light can be defined as having the specified cross-section (e.g., a particular profile) at the substrate, where the constructive and/or destructive interference occurs. With an amplitude spatial light modulator, the impinging light can be defined as having the specified cross-section (e.g., a particular profile) at the substrate, and/or at any point between the amplitude spatial light modulator and the substrate.

The selection of a shape of illumination light (e.g., one or more of the shapes 416) can affect the intensity profile and/or irradiance profile of the light impinging at the sample. For example, this can apply when the spatial light modulator is a phase spatial light modulator. In some implementations, when the shapes 416E are being used, this causes a resulting level of brightness in the illuminated portion of the substrate (e.g., with uniform illumination density). By changing the analysis system to instead form another shape (e.g., the shape 416D), an increased brightness of the illumination light can be obtained at the sample compared to before the change. As such, the shape of the illumination pattern can be selected at least in part based on the desired level of illumination light brightness. An optical system such as the system 400 can be subject to transmission losses that affect the resulting power and/or brightness of the light. In some implementations, transmission loss can occur at the spatial light modulator 402. For example, the transmission loss(es) can be taken into account in selecting the light source 404 for the system 400, and/or in adjusting the settings of the light source 404.

An arrow 418 here connects the substrate 412 and the spatial light modulator 402 to each other. In some implementations, the arrow 418 schematically illustrates that the system 400 can feature shape selection of illumination light. In some implementations, the system 400 can detect an identifier at the substrate 412, the identifier relating to a sensor included in the substrate. Based on the detection, the system 400 can control the spatial light modulator 402 to generate one or more shapes of illuminating light at the substrate 412. For example, more than one area of illumination light can be provided when the substrate 412 has more than one sensor. The detected identifier can include an RFID tag, and/or a visual code, to name just two examples.

In some implementations, the system 400 includes a wireless sensor and/or an optical sensor that detects the identifier (e.g., by receiving a radio transmission or by scanning a visual code such as a barcode). The sensor can output one or more signals indicative of data encoded in the identifier. The system 400 can use the signal(s) from the sensor to control the spatial light modulator 402. For example, the spatial light modulator 402 can be controlled to generate an illumination light profile based on the data encoded in the identifier.

In some implementations, the identifier at the substrate 412 may not relate to any sensor(s) of the substrate 412. For example, the substrate 412 may not include any sensor, and/or the system 400 may include at least one sensor 420 to detect the emission(s). Imaging light can propagate from the substrate 412 by way of the lens 410 (e.g., an objective lens) and the mirror 408 (e.g., a dichroic mirror) toward the sensor 420 for detection. The identifier at the substrate 412 can indicate a setting or other characteristic to be applied to the sensor(s) 420 in the system 400. The identifier can indicate how many areas of the sensor(s) 420 should be used for detection, and/or can define the position of such area(s) at the sensor(s) 420, to name just a few examples.

In some implementations, the arrow 418 can also or instead schematically illustrate that the system 400 can feature real-time feedback regarding the illumination light as detected at the substrate 412. The uniformity of the illuminating light at the substrate 412 can be relevant to the quality and/or efficiency of the sample analysis. As such, the spatial light modulator 402 can be controlled in a way intended to provide a certain level of uniformity of the illumination light at the substrate 412. However, due to unknown or unavoidable characteristics of the optical path, some amount of non-uniform signal response can be detected at the substrate 412. The system can therefore perform a comparison based on the illumination light and the response signal from the imaging. For example, the system 400 can create the inverse of the imaging pattern with the excitation light, and multiply the inverse with the obtained imaging. In principle, the outcome of such multiplication is a true flat signal response. However, if the product resulting from the multiplication differs from the true flat signal response, this can suggest making an adjustment of the spatial light modulator 402 (and/or of the light source 404). As such, the real-time feedback can help improve the efficiency and/or quality of the analysis performed by the system 400.

In some implementations, the system 400 includes a pattern inverting component that generates an inverse of the imaging pattern with the excitation light. For example, the pattern inverting component can be configured to perform a transformation that is the inverse of the transform (e.g., a phase shift) that is performed by a phase spatial light modulator. In some implementations, the response signal of the obtained imaging can be obtained. For example, this can be done using a sensor located at the sample substrate, and/or by a sensor elsewhere in the system. In some implementations, the system 400 includes a pattern multiplication component. The pattern multiplication component can perform a multiplication based on the generated inverse and the response signal. For example, the pattern multiplication component can obtain the Fourier transforms of the inverse and the response signal and multiply their respective coefficients with each other. If the factors involved in the pattern multiplication are truly each other's inverses, then the product can be expected to be a true flat signal (e.g., to have unity value everywhere). However, when there are discrepancies between the inverse and the response signal, the product may not be flat (e.g., some values are greater or smaller than one). When one or more non-flat (e.g., non-unity) values appear in the product, the spatial light modulator 402 and/or the light source 404 can be adjusted to counteract the situation.

The above examples illustrate an example of a system for analysis of a sample at a substrate (e.g., the substrate 412). Such a system (e.g., the system 400) can include: a light source (e.g., the light source 404) to generate first light; and a spatial light modulator (e.g., the spatial light modulator 402) to form second light from the first light. The substrate can include at least one sensor (e.g., the sensor layer 306 in FIG. 3) to detect an emission by, or associated with, the sample. At the substrate the second light can form a shape (e.g., any of the shapes 416) selected based on the at least one sensor. The second light can illuminate an area of the substrate corresponding to the shape.

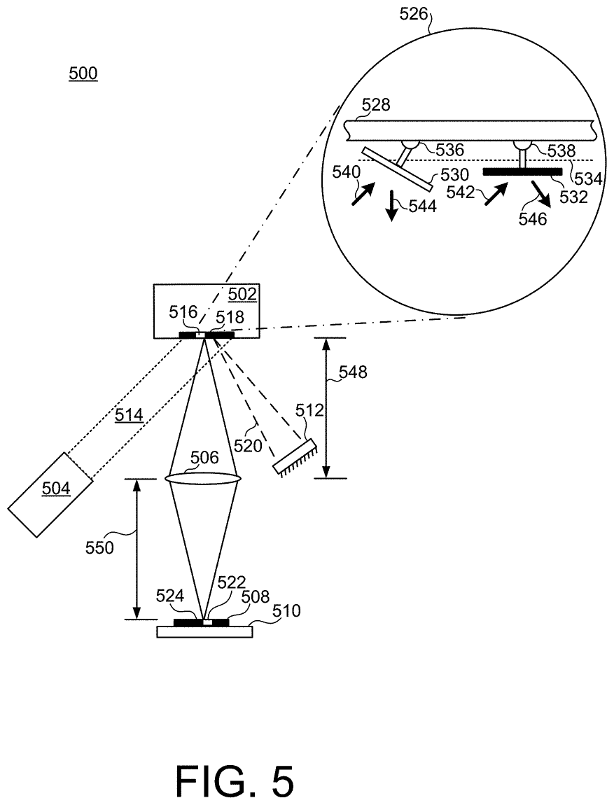

FIG. 5 shows an example of a system 500 with an amplitude spatial light modulator 502. The system 500, and/or the amplitude spatial light modulator 502, can be used with one or more other examples described elsewhere herein. In this example, the amplitude spatial light modulator 502 operates by reflecting light. In some implementations, the amplitude spatial light modulator 502 can be transmissive. The system 500 includes a light source 504 (e.g., one or more lasers and/or LED devices), and at least one projection lens 506 that directs light toward a sample 508 at a substrate 510. In some implementations, the system 500 includes a finite conjugate lens. In some implementations, the system 500 includes a tube lens and a microscope objective, with a collimated area (e.g., one or more collimators) in between. The system 500 here also includes a beam dump 512. As used herein, a beam dump is one or more components configured for absorbing one or more types of light. In some implementations, light can be directed toward the beam dump to prevent the light from propagating elsewhere and potentially interfering with another component in an unwanted way. For example, the beam dump can be designed to absorb the energy of photons in the light with little or no reflection.

In operation, the light source 504 directs light 514 toward the amplitude spatial light modulator 502. The system 500 controls the amplitude spatial light modulator 502 to create at least one light area 516 and a dark area 518 thereon. The light area 516 corresponds to reflection of a portion of the light 514 toward the projection lens 506. For example, this can result in a light area 522 appearing at the sample 508. The dark area 518 corresponds to reflection of a portion of the light 514 elsewhere than toward the projection lens 506. For example, the dark area 518 can reflect light 520 toward the beam dump 512. This can result in a dark area 524 appearing at the sample 508. At the sample 508, the light area 522 and the dark area 524 (and optionally one or more other light/dark areas not shown) can form at least one shape (e.g., one or more of the shapes 416 in FIG. 4). The present example mentions two areas at the amplitude spatial light modulator 502 (the light area 516 and the dark area 518), and two areas at the sample 508 (the light area 522 and the dark area 524), for simplicity. In some implementations, more or fewer areas can be used. In some implementations, the substrate 510 includes one or more sensors. In some implementations, the system 500 includes one or more sensors located elsewhere than at the substrate 510.

The amplitude spatial light modulator 502 can operate according to at least one of multiple approaches for spatially modulating the light 514. In some implementations, the amplitude spatial light modulator 502 can include a deformable mirror. As used herein, a deformable mirror includes any device having a surface that is reflective to at least one type of light, wherein the surface can be controllably deformed to give the reflected light one or more characteristics different from that if the incident light. For example, a deformable mirror can operate according to one or more of the following techniques: independent flat mirror segments are movable in forward and rearward directions to accomplish a phase shift; a thin deformable membrane is controlled by discrete actuators at its back side; a continuous reflective surface is motioned by the individual strokes of magnetic actuators; micro-electrical-mechanical mirrors are controlled by actuators; a thin conductive and reflective membrane is stretched over a solid flat frame, and is deformed electrostatically by applied voltages; a piezoelectric or electrostrictive material is patterned with an electrode structure to which voltage is applied; or ferromagnetic particles are suspended in a liquid carrier and subjected to an external magnetic field that shapes the surface.

In some implementations, the deformable mirror can be deformed in one or more parts to provide the light area 516 and the dark area 518. In some implementations, the amplitude spatial light modulator 502 can include mirrors that are orientable to assume any of at least two different states. Here, a circle 526 schematically shows a substrate 528 that is part of the amplitude spatial light modulator 502. The substrate 528 can be provided with at least mirrors 530 and 532. More mirrors than shown can be used. For example, the mirrors 530 and 532 can be part of an array of micromirrors in a micro-electrical-mechanical system (MEMS). In the present example, the mirror 530 will form at least part of the light area 516 and is therefore shown in white. In this example, the mirror 532 will form at least part of the dark area 518 and is therefore shown in black. The mirrors 530 and 532 can be made of the same material as each other. The orientation of the mirrors 530 and 532 can be defined relative to any reference, such as a line 534 indicated in the illustration. The position of the mirror 530 relative to the line 534 can be controlled by a mechanism 536. The position of the mirror 532 relative to the line 534 can be controlled by a mechanism 538. The mechanisms 536 and 538 can operate according to any of multiple MEMS technological approaches. In some implementations, each of the mechanisms 536 and 538 includes a torsion hinge controlled by a CMOS memory element so as to control the position of the corresponding one of the mirrors 530 and 532 using one or more springs.

In operation, light 540 and light 542 can reach the amplitude spatial light modulator 502 as part of the light 514. The lights 540 and 542 can be referred to as first light directed at the amplitude spatial light modulator 502. The mirror 530 can reflect light 544 from the light 540. The light 544 can be referred to as second light being directed toward the projection lens 506. The mirror 532 can reflect light 546 from the light 542, the light 546 forming at least part of the light 520. The light 546 can be referred to as third light being directed toward the beam dump 512.

The relative positions of the amplitude spatial light modulator 502, the projection lens 506, and the substrate 510 to each other can be selected based on one or more considerations. Here, a distance 548 between the amplitude spatial light modulator 502 and the projection lens 506 is indicated. Also, a distance 550 between the projection lens 506 and the sample 508 is indicated. To provide about unity, or 1:1, magnification in the system 500, the distances 548 and 550 can be at least substantially equal to each other. For example, the distances 548 and 550 can both be about equal to 2f where f represents the focal length of the system 500. The distances 548 and 550 can have other lengths, for example so that they are not equal.

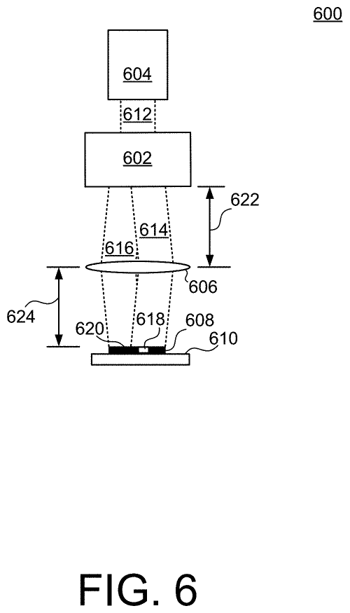

FIG. 6 shows an example of a system 600 with a phase spatial light modulator 602. The system 600, and/or the phase spatial light modulator 602, can be used with one or more other examples described elsewhere herein. In this example, the phase spatial light modulator 602 operates by transmitting light. In some implementations, the phase spatial light modulator 602 can be reflective. The system 600 includes a light source 604 (e.g., one or more lasers and/or LED devices), and at least one projection lens 606 that directs light toward a sample 608 at a substrate 610. In some implementations, the phase spatial light modulator 602 can be located at an entrance pupil of the projection lens 606 (e.g., directly or using an optical relaying system). In some implementations, the phase spatial light modulator 602 can generate the Fourier transform of the pattern to be formed at the sample 608. For example, an iterative algorithm can be used to calculate the phase pattern given the capabilities of the phase spatial light modulator 602 (e.g., its resolution and/or the phase shift that can be achieved).

In operation, the light source 604 directs light 612 toward the phase spatial light modulator 602. The system 600 controls the phase spatial light modulator 602 to form light from the light 612. Here, lights 614 and 616 from the phase spatial light modulator 602 are schematically illustrated. For example, the phase spatial light modulator 602 can phase shift one of the lights 614 and 616 relative to the other. The phase spatial light modulator 602 can be based on liquid crystals. In some implementations, a liquid crystal arrangement can have different refractive indices depending on the polarization and/or propagation direction of light (i.e., to be birefringent). The liquid crystals can be controlled (e.g., by applying a voltage across a thickness of the liquid crystal layer) to change the effective refractive index for one portion of light compared to another portion of light for which a different voltage (or no voltage) was applied. The changed refractive index applied to some of the light causes a phase shift of at least one portion of the light relative to another portion of the light. As such, the light formed by the spatial light modulator 602 can be characterized by having one or more phase shifts.

Phase shifting can result in constructive or destructive interference at the sample 608. For example, a light area 618 appearing at the sample 608 can result from constructive interference between the lights 614 and 616. As another example, a dark area 620 appearing at the sample 608 can result from destructive interference between the lights 614 and 616. At the sample 508, the light area 618 and the dark area 620 (and optionally one or more other light/dark areas not shown) can form at least one shape (e.g., one or more of the shapes 416 in FIG. 4). In some implementations, the substrate 610 includes one or more sensors. In some implementations, the system 600 includes one or more sensors located elsewhere than at the substrate 610.

The relative positions of the phase spatial light modulator 602, the projection lens 606, and the substrate 610 to each other can be selected based on one or more considerations. Here, a distance 622 between the phase spatial light modulator 602 and the projection lens 606 is indicated. Also, a distance 624 between the projection lens 606 and the sample 608 is indicated. To provide about unity, or 1:1, magnification in the system 600, the distances 622 and 624 can be at least substantially equal to each other. For example, the distances 622 and 624 can both be about equal to 1f, where f represents the focal length of the system 600. The distances 622 and 624 can have other lengths, for example where they are not equal.

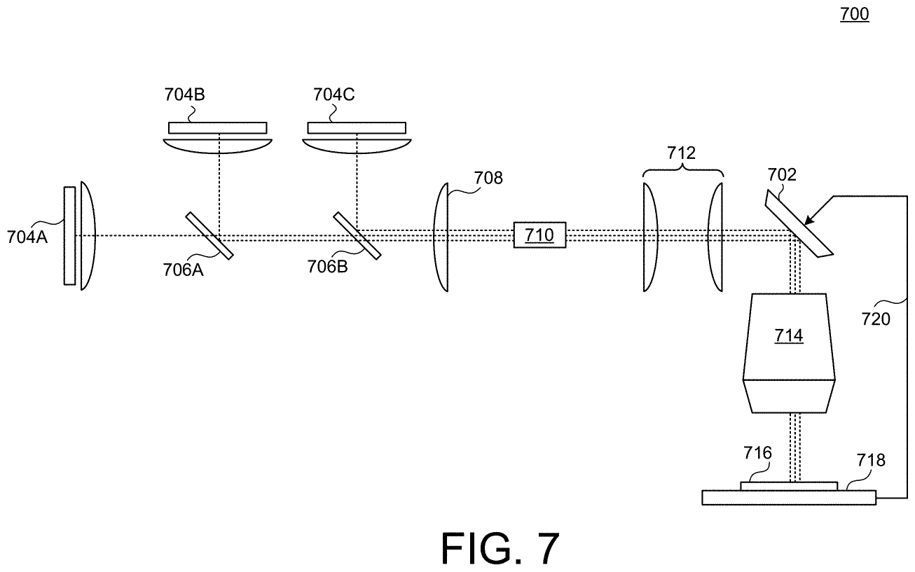

FIG. 7 shows an example of a system 700 with real-time feedback to a spatial light modulator 702. The system 700, and/or the spatial light modulator 702, can be used with one or more other examples described elsewhere herein. The system 700 can include one or more light sources. Here, LED arrays 704A-704C are shown. Each of the LED arrays 704A-704C can generate light of one or more wavelength bands. For example, blue, green and/or red light can be generated. Light from any of the LED arrays 704A-704C can pass through one or more lenses, for example as shown. Light from some or all of the LED arrays 704A-704C can be brought into a common optical path. For example, a dichroic filter 706A and/or a dichroic filter 706B can be used. The light from one or more of the LED arrays 704A-704C can be condensed, for example using at least one condenser lens 708. The light from one or more of the LED arrays 704A-704C can be collimated, for example using at least one collimator 710. Other optics can be applied to the light from one or more of the LED arrays 704A-704C, as here schematically illustrated by lenses 712. The lenses 712 can direct at least some of the light from one or more of the LED arrays 704A-704C toward the spatial light modulator 702.

The light from the spatial light modulator 702 can propagate toward a projection lens 714. In this example, the spatial light modulator 702 operates by reflecting at least some of the light. In some implementations, the spatial light modulator 702 can operate by transmitting at least some of the light. The projection lens 714 can direct the light toward a sample 716 at a substrate 718. The substrate 718 can include one or more sensors to detect one or more types of emission from (or associated with) the sample 716 in response to illumination light. For example, fluorescent light can be detected in response to excitation light. The sensor of the substrate 718 can be used for providing real-time feedback to the spatial light modulator 702, as schematically indicated by an arrow 720. In some implementations, the real-time feedback can be based on comparison involving an inverse of the imaging signal captured at the sensor. The inverse can be multiplied with the illumination pattern as defined by the spatial light modulator 702. In some implementations, if the result is not a flat signal, the system 700 can adjust one or more parameters (e.g., by controlling the spatial light modulator 702). For example, if the non-flat signal indicates that the illumination footprint of the spatial light modulator 702 causes light to impinge in a particular area of the substrate (or outside of the substrate), the footprint can be adjusted so as to not generate light in that area. As another example, if the non-flat signal indicates that the illumination footprint of the spatial light modulator 702 causes little or no light to impinge in a particular area of the substrate, the footprint can be adjusted so as to generate light in that area. For example, the above control operations can involve adjustments of an amplitude spatial light modulator (e.g., by making more or fewer mirrors light or dark). As another example, the above control operations can involve adjustments of a phase spatial light modulator (e.g., to adjust the transformation to cause constructive interference to occur in a particular area, or to cause destructive interference to occur in a particular area). In some implementations, the system 700 includes one or more sensors located elsewhere than at the substrate 718.

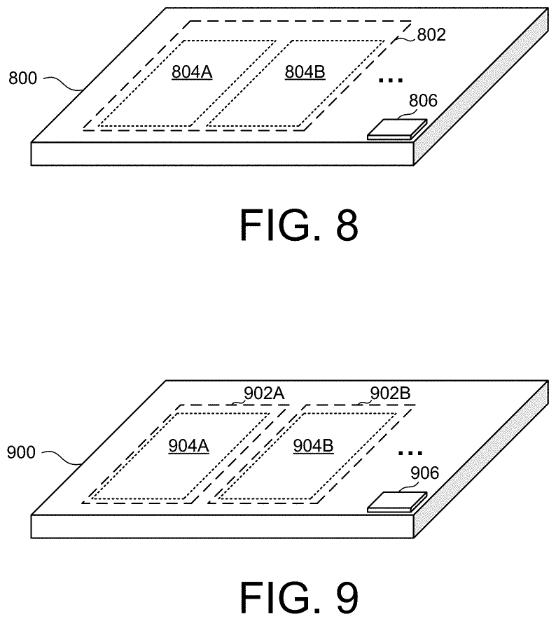

FIG. 8 shows an example of a substrate 800 having a flow cell 802 and sensors 804A-804B. The substrate 800, the flow cell 802, and/or the sensors 804A-804B can be used with one or more other examples described elsewhere herein. The flow cell 802 is here schematically illustrated using a dashed outline and can include one or more channels for flowing a fluid (e.g., reagent and/or liquid with fluorescing dye) in contact with the sample (not shown). The present position of the flow cell 802, being at the “top” of the substrate 800, is shown for illustrative purposes only. The substrate 800 can include one or more other flow cells (not shown) in addition to the flow cell 802.

The sensors 804A-804B are here schematically illustrated using a dashed outline and can include one or more devices designed for detecting emissions from (or associated with) the samples. The sensors 804A-804B are here covered by the flow cell 802. The present position of the sensors 804A-804B, being “below” the flow cell 802 in the substrate 800, is shown for illustrative purposes only. The substrate 800 can include one or more other sensors (not shown) in addition to the sensors 804A-804B.

The substrate 800 can include an identifier 806 relating to the sensors 804A-804B. In some implementations, the identifier 806 can indicate at least the number of sensors (e.g., one or more) included in the substrate 800. A system can detect the identifier 806 and select a shape of the illumination light (e.g., excitation light) based on the sensors 804A-804B. In some implementations, the identifier 806 can be provided using an RFID tag and/or a visual code.

In some implementations, the identifier 806 may not relate to any sensor(s) of the substrate 800. For example, the substrate 800 may not include any sensor, and/or a system where the substrate 800 is used may include one or more sensors to detect the emission(s). The identifier 806 can indicate a setting or other characteristic to be applied to such sensor(s) in the system. The identifier can indicate how many areas of the sensor(s) should be used for detection, and/or the position of such area(s) at the sensor(s), to name just a few examples.

FIG. 9 shows an example of a substrate 900 having flow cells 902A-902B and sensors 904A-904B. The substrate 900, flow cells 902A-902B, and/or sensors 904A-904B can be used with one or more other examples described elsewhere herein. The flow cells 902A-902B are here schematically illustrated using a dashed outline and can each include one or more channels for flowing a fluid (e.g., reagent and/or liquid with fluorescing dye) in contact with the sample (not shown). The present position of the flow cells 902A-902B, being at the “top” of the substrate 900, is shown for illustrative purposes only. The substrate 900 can include one or more other flow cells (not shown) in addition to the flow cells 902A-902B.

The sensors 904A-904B are here schematically illustrated using a dashed outline and can each include one or more devices designed for detecting emissions from (or associated with) the samples. Here, the sensor 904A is covered by the flow cell 902A. The sensor 904B is covered by the flow cell 902B. The present position of the sensors 904A-904B, being “below” the respective flow cells 902A-902B in the substrate 900, is shown for illustrative purposes only. The substrate 900 can include one or more other sensors (not shown) in addition to the sensors 904A-904B.

The substrate 900 can include an identifier 906 relating to the sensors 904A-904B. In some implementations, the identifier 906 can indicate at least the number of sensors (e.g., two or more) included in the substrate 900. A system can detect the identifier 906 and select a shape of the illumination light (e.g., excitation light) based on the sensors 904A-904B. In some implementations, the identifier 906 can be provided using an RFID tag and/or a visual code.

In some implementations, the identifier 906 may not relate to any sensor(s) of the substrate 900. For example, the substrate 900 may not include any sensor, and/or a system where the substrate 900 is used may include one or more sensors to detect the emission(s). The identifier 906 can indicate a setting or other characteristic to be applied to such sensor(s) in the system. The identifier can indicate how many areas of the sensor(s) should be used for detection, and/or the position of such area(s) at the sensor(s), to name just a few examples.

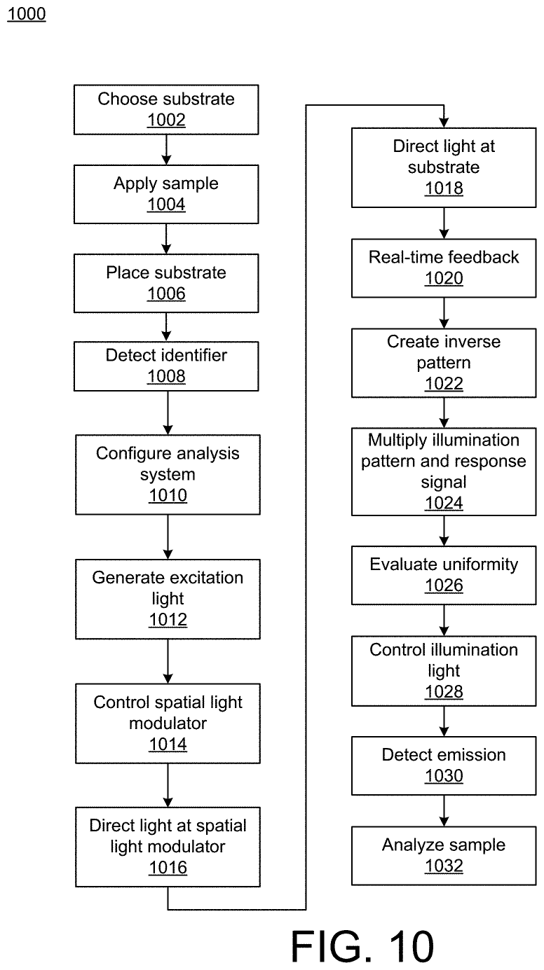

FIG. 10 shows an example of a method 1000. The method 1000 can be used with one or more other examples described elsewhere herein. More or fewer operations can be performed. Two or more operations can be performed in aa different order unless otherwise indicated.

At operation 1002, a substrate can be selected. This selection can be made by a person involved in the analysis process and can take into account the type of sample and/or the kind(s) of emission to be detected. The selection can be made from among a number of types of substrates. For example, the substrate 800 (FIG. 8) or the substrate 900 (FIG. 9) can be selected.

At operation 1004, at least one sample can be applied to the selected substrate. In some implementations, the sample includes genetic material that is to be sequenced. For example, preparation of a sample of genetic material can include clustering of the sample, including, but not limited to, solid-phase amplification of template molecules.

At operation 1006, the substrate can be placed in position relative to the analysis system. In some implementations, this can involve placing the substrate inside an enclosure of the system, such as on a translatable stage where thermal control and/or fluid treatment can be performed.

At operation 1008, an identifier of the substrate can be detected. In some implementations, the identifier can relate to the sensor(s) of the substrate. In some implementations, the identifier can relate to one or more sensors located elsewhere than at the substrate. In some implementations, detecting the identifier can involve receiving a signal from an RFID tag and/or reading a visual code.

At operation 1010, the analysis system can be configured. For example, the characteristics of the illumination light or the duration of its application to the sample can be defined. As another example, the characteristics of the detection to be performed by the sensor or the duration of its operation can be defined. Other configurations can be performed additionally or alternatively.

At operation 1012, illumination light (e.g., excitation light) can be generated by a light source. The generated light can be directed at the spatial light modulator.

At operation 1014, the spatial light modulator can be controlled to form light from the light received as part of the operation 1012. The spatial light modulator can be controlled to form the light so as to be directed at the sample. In some implementations, this can involve controlling the spatial light modulator to form light of one or more selected shapes. For example, the number of shapes to be formed by the light can be selected based on the number of sensors of the substrate.

At operation 1016, light can be directed at the spatial light modulator. For example, one or more optics components (e.g., a lens, collimator, and/or prism can be used).

At operation 1018, light from the spatial light modulator can be directed at the substrate. In some implementations, an amplitude spatial light modulator can direct some light toward the sample at the substrate, and can direct other light elsewhere (e.g., into a beam dump). As another example, a phase spatial light modulator can change the phase of at least a portion of the received light.

At operation 1020, performance of real-time feedback can be triggered. For example, this can be done during a pre-analysis procedure or at an initial stage of the analysis procedure.

At operation 1022, an inverse pattern can be generated. In some implementations, this involves using an output from the excitation optics. For example, an inverse of the imaging pattern with the excitation light can be generated.

At operation 1024, the generated inverse pattern can be multiplied with the response signal of the obtained imaging. For example, this can involve pattern multiplication using respective Fourier transforms.

At operation 1026, the outcome of the multiplication in the operation 1024 can be taken into account in evaluating uniformity of the illumination light. For example, such evaluation can be part of the basis for deciding whether to adjust the analysis system.

At operation 1028, the illuminating light can be controlled. For example, this can involve changing or adjusting one or more characteristics of the analysis system based on the evaluation in the operation 1026.

At operation 1030, at least one emission from (or associated with) the sample can be detected. In some implementations, the emission(s) can be detected by a sensor that is included in the substrate. For example, the emission detection can involve imaging of the sample by way of registering fluorescent light generated in response to excitation light.

At operation 1032, the sample can be analyzed. One or more aspects or characteristics of the sample can be determined or estimated based on the emission detection. For example, with a sample of genetic material sequencing of the sample can be performed to determine its primary structure.

FIG. 11 illustrates an example architecture of a computing device 1100 that can be used to implement aspects of the present disclosure, including any of the systems, apparatuses, and/or techniques described herein, or any other systems, apparatuses, and/or techniques that may be utilized in the various possible embodiments.

The computing device illustrated in FIG. 11 can be used to execute the operating system, application programs, and/or software modules (including the software engines) described herein.

The computing device 1100 includes, in some embodiments, at least one processing device 1102 (e.g., a processor), such as a central processing unit (CPU). A variety of processing devices are available from a variety of manufacturers, for example, Intel or Advanced Micro Devices. In this example, the computing device 1100 also includes a system memory 1104, and a system bus 1106 that couples various system components including the system memory 1104 to the processing device 1102. The system bus 1106 is one of any number of types of bus structures that can be used, including, but not limited to, a memory bus, or memory controller; a peripheral bus; and a local bus using any of a variety of bus architectures.

Examples of computing devices that can be implemented using the computing device 1100 include a desktop computer, a laptop computer, a tablet computer, a mobile computing device (such as a smart phone, a touchpad mobile digital device, or other mobile devices), or other devices configured to process digital instructions.

The system memory 1104 includes read only memory 1108 and random access memory 1110. A basic input/output system 1112 containing the basic routines that act to transfer information within computing device 1100, such as during start up, can be stored in the read only memory 1108.

The computing device 1100 also includes a secondary storage device 1114 in some embodiments, such as a hard disk drive, for storing digital data. The secondary storage device 1114 is connected to the system bus 1106 by a secondary storage interface 1116. The secondary storage device 1114 and its associated computer readable media provide nonvolatile and non-transitory storage of computer readable instructions (including application programs and program modules), data structures, and other data for the computing device 1100.

Although the example environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. For example, a computer program product can be tangibly embodied in a non-transitory storage medium. Additionally, such computer readable storage media can include local storage or cloud-based storage.

A number of program modules can be stored in secondary storage device 1114 and/or system memory 1104, including an operating system 1118, one or more application programs 1120, other program modules 1122 (such as the software engines described herein), and program data 1124. The computing device 1100 can utilize any suitable operating system, such as Microsoft Windows™, Google Chrome™ OS, Apple OS, Unix, or Linux and variants and any other operating system suitable for a computing device. Other examples can include Microsoft, Google, or Apple operating systems, or any other suitable operating system used in tablet computing devices.

In some embodiments, a user provides inputs to the computing device 1100 through one or more input devices 1126. Examples of input devices 1126 include a keyboard 1128, mouse 1130, microphone 1132 (e.g., for voice and/or other audio input), touch sensor 1134 (such as a touchpad or touch sensitive display), and gesture sensor 1135 (e.g., for gestural input). In some implementations, the input device(s) 1126 provide detection based on presence, proximity, and/or motion. In some implementations, a user may walk into their home, and this may trigger an input into a processing device. For example, the input device(s) 1126 may then facilitate an automated experience for the user. Other embodiments include other input devices 1126. The input devices can be connected to the processing device 1102 through an input/output interface 1136 that is coupled to the system bus 1106. These input devices 1126 can be connected by any number of input/output interfaces, such as a parallel port, serial port, game port, or a universal serial bus. Wireless communication between input devices 1126 and the input/output interface 1136 is possible as well, and includes infrared, BLUETOOTH® wireless technology, 802.11a/b/g/n, cellular, ultra-wideband (UWB), ZigBee, or other radio frequency communication systems in some possible embodiments, to name just a few examples.

In this example embodiment, a display device 1138, such as a monitor, liquid crystal display device, light-emitting diode display device, projector, or touch sensitive display device, is also connected to the system bus 1106 via an interface, such as a video adapter 1140. In addition to the display device 1138, the computing device 1100 can include various other peripheral devices (not shown), such as speakers or a printer.

The computing device 1100 can be connected to one or more networks through a network interface 1142. The network interface 1142 can provide for wired and/or wireless communication. In some implementations, the network interface 1142 can include one or more antennas for transmitting and/or receiving wireless signals. When used in a local area networking environment or a wide area networking environment (such as the Internet), the network interface 1142 can include an Ethernet interface. Other possible embodiments use other communication devices. For example, some embodiments of the computing device 1100 include a modem for communicating across the network.

The computing device 1100 can include at least some form of computer readable media. Computer readable media includes any available media that can be accessed by the computing device 1100. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 1100.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 11 is also an example of programmable electronics, which may include one or more such computing devices, and when multiple computing devices are included, such computing devices can be coupled together with a suitable data communication network so as to collectively perform the various functions, methods, or operations disclosed herein.

The terms “substantially” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. Also, when used herein, an indefinite article such as “a” or “an” means “at least one.”

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

In addition, the logic flows depicted in the figures do not require the particular order shown, or sequential order, to achieve desirable results. In addition, other processes may be provided, or processes may be eliminated, from the described flows, and other components may be added to, or removed from, the described systems. Accordingly, other implementations are within the scope of the following claims.

While certain features of the described implementations have been illustrated as described herein, many modifications, substitutions, changes and equivalents will now occur to those skilled in the art. It is, therefore, to be understood that appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

What is claimed is:

1. A system for analysis of a sample at a substrate, the system comprising:
a light source to generate first light; and
a spatial light modulator to form second light from the first light, wherein the substrate includes at least one sensor to detect an emission emitted based on the second light, wherein at the substrate the second light forms a shape selected based on the at least one sensor, wherein the second light illuminates an area of the substrate corresponding to the shape.

2. The system of claim 1, wherein the spatial light modulator is a transmissive spatial light modulator.

3. The system of claim 1, wherein the spatial light modulator is a reflective spatial light modulator.

4. The system of claim 1, wherein the spatial light modulator is an amplitude spatial light modulator.

5. The system of claim 4, wherein the amplitude spatial light modulator includes mirrors that are orientable, and wherein the amplitude spatial light modulator orients at least a first mirror of the mirrors to form the second light.

6. The system of claim 5, further comprising a beam dump, wherein the amplitude spatial light modulator orients at least a second mirror of the mirrors to direct third light at the beam dump, the third light being part of the first light.

7. The system of claim 1, wherein the spatial light modulator is a phase spatial light modulator.

8. The system of claim 1, wherein the area of the substrate includes a polygon.

9. The system of claim 8, wherein the second light illuminates multiple areas of the substrate, each of the multiple areas being a polygon area.

10. The system of claim 1, wherein the area of the substrate includes an ellipse.

11. The system of claim 10, wherein the second light illuminates multiple areas of the substrate, each of the multiple areas being an ellipse area.

12. The system of claim 1, wherein the second light illuminates multiple areas of the substrate.

13. The system of claim 12, wherein at least two of the multiple areas have different shapes from each other.

14. The system of claim 1, wherein the sensor comprises a complementary metal oxide semiconductor device.

15. The system of claim 1, wherein the substrate includes a flow cell for the sample.

16. The system of claim 15, wherein the substrate includes multiple sensors covered by the flow cell.

17. The system of claim 15, wherein the substrate includes multiple flow cells.

18. The system of claim 17, wherein the substrate includes multiple sensors, and wherein each of the multiple flow cells covers at least a corresponding one of the multiple sensors.

19. The system of claim 1, wherein the substrate further includes an identifier relating to the sensor, wherein the system detects the identifier at the substrate, and wherein the shape is selected based on the detected identifier.

20. The system of claim 19, wherein the identifier comprises at least one of a radiofrequency identification tag or a visual code.

21. The system of claim 1, wherein the emission comprises fluorescent light.

22. A method comprising:
directing first light at a spatial light modulator in a system configured for analysis of a sample at a substrate, wherein the substrate includes at least one sensor;
controlling the spatial light modulator to form second light from the first light;
directing the second light at the substrate, wherein the second light illuminates an area of the substrate having a shape selected based on the at least one sensor; and
detecting an emission emitted based on the second light.

23. The method of claim 22, wherein the spatial light modulator is an amplitude spatial light modulator, wherein the amplitude spatial light modulator includes mirrors that are orientable, and wherein controlling the amplitude spatial light modulator to form the second light comprises orienting at least a first mirror of the mirrors.

24. The method of claim 23, wherein the first mirror is oriented to assume one of at least two states.

25. The method of claim 23, further comprising orienting at least a second mirror of the mirrors to direct third light at a beam dump, the third light being part of the first light.

26. The method of claim 22, wherein the spatial light modulator is a phase spatial light modulator, and wherein controlling the phase spatial light modulator to form the second light comprises changing an intensity profile or an irradiance profile of the second light.

27. The method of claim 26, wherein changing the intensity profile or the irradiance profile comprises changing the second light from illuminating a plurality of areas of the substrate to illuminating at least one area that is fewer than the plurality of areas, and wherein a brightness is increased in the at least one area.

28. The method of claim 22, wherein the second light illuminates multiple areas of the substrate.

29. The method of claim 28, wherein at least two of the multiple areas have different shapes from each other.

30. The method of claim 22, wherein the substrate further includes an identifier relating to the sensor, the method further comprising detecting, by the system, the identifier at the substrate, and wherein the shape is selected based on the detected identifier.

31. The method of claim 30, wherein detecting the identifier comprises at least one of receiving a signal from a radiofrequency identification tag, or reading a visual code.

32. The method of claim 22, further comprising:
providing real-time feedback regarding the second light; and
controlling the spatial light modulator using the real-time feedback.

33. The method of claim 32, wherein providing the real-time feedback comprises:
creating an inverse of an imaging pattern of the second light; and
multiplying the inverse with imaging obtained at the substrate.

34. The method of claim 22, wherein the emission comprises fluorescent light.
